(19) United States
(12) Patent Application Publication
(10) Pub. No.: US 2022/0218812 A1
(43) Pub. Date: Jul. 14, 2022

(54) METHODS OF TREATING PATIENTS WITH AN IMMUNOGENIC COMPOSITION THAT PROTECTS AGAINST S. PNEUMONIAE SEROTYPE 29

(71) Applicant: Merck Sharp & Dohme Corp., Rahway, NJ (US)

(72) Inventors: Jian He, Blue Bell, PA (US); Robin M. Kaufhold, Lansdale, PA (US); Julie M. Skinner, Phoenixville, PA (US); Jinfu Xie, Haveford, PA (US)

(73) Assignee: Merck Sharp & Dohme Corp., Rahway, NJ (US)

(21) Appl. No.: 17/614,876

(22) PCT Filed: Jun. 1, 2020
(86) PCT No.: PCT/US2020/035511

§ 371 Date: Nov. 29, 2021

Related U.S. Application Data
(60) Provisional application No. 62/857,534, filed on Jun. 5, 2019.

(51) Int. Cl. A61K 39/09; A61P 31/04; A61K 47/64

(52) U.S. Cl. CPC A61K 39/092; A61P 31/04; A61K 2039/575; A61K 47/646; A61K 47/6415

(57) ABSTRACT

The present invention provides methods for treating patients by administering an immunogenic multivalent pneumococcal polysaccharide-protein conjugate vaccine which comprises a S. pneumoniae serotype 35B polysaccharide-protein conjugate, does not comprise a S. pneumoniae serotype 29 polysaccharide-protein conjugate, and provides protection against S. pneumoniae serotype 29.

### FIELD OF INVENTION

The present invention provides methods for treating patients by administering an immunogenic multivalent pneumococcal polysaccharide-protein conjugate vaccine composition which comprises a serotype 35B polysaccharide-protein conjugate, does not comprise a serotype 29 polysaccharide-protein conjugate, and provides protection against S. pneumoniae serotype 29.

### BACKGROUND OF THE INVENTION

Streptococcus pneumoniae (S. pneumoniae) is a Gram-positive bacterium and the most common cause of invasive bacterial disease (such as pneumonia, bacteremia, meningitis and otitis media) in infants and young children. Pneumococcus is encapsulated with a chemically linked polysaccharide which confers serotype specificity. There are over 90 known serotypes of pneumococci, and the capsule is the principle virulence determinant for pneumococci, as the capsule not only protects the inner surface of the bacteria from complement, but is itself poorly immunogenic. Polysaccharides are T-cell independent antigens, and, in most cases, cannot be processed or presented on MHC molecules to interact with T-cells. They can however, stimulate the immune system through an alternate mechanism which involves cross-linking of surface receptors on B cells.

The multivalent pneumococcal polysaccharide vaccines that have been licensed for many years have proved valuable in preventing pneumococcal disease in adults, particularly, the elderly and those at high-risk. However, infants and young children respond poorly to unconjugated pneumococcal polysaccharides. The pneumococcal conjugate vaccine, Prevnar®, containing the 7 most frequently isolated serotypes (4, 6B, 9V, 14, 18C, 19F and 23F) causing invasive pneumococcal disease in young children and infants at the time, was first licensed in the United States in February 2000. Following universal use of Prevnar® in the United States, there has been a significant reduction in invasive pneumococcal disease in children due to the serotypes present in Prevnar®. See Centers for Disease Control and Prevention, MMWR Morb Mortal Wkly Rep 2005, 54(36):893-7. However, there are limitations in serotype coverage with Prevnar® in certain regions of the world and some evidence of certain emerging serotypes in the United States (for example, 19A and others). See O'Brien et al., 2004, Am J Epidemiol 159:634-44; Whitney et al., 2003, N Engl J Med 348:1737-46; Kyaw et al., 2006, N Engl J Med 354:1455-63; Hicks et al., 2007, J Infect Dis 196:1346-54; Traore et al., 2009, Clin Infect Dis 48:S181-S189. Other multivalent pneumococcal polysaccharide-protein conjugate vaccines are known (US 2006/0228380, CN 101590224, and US 2011/0195086, among others).

Immune interference has been observed in multivalent pneumococcal polysaccharide-protein conjugate vaccines (e.g. lower protection for serotype 3 in GSK's PCV-11) and lower response rates to serotype 6B in Pfizer's PCV-13 (PREVNAR® 13). See Prymula et al., 2006, Lancet 367:740-48 and Kieninger et al., Safety and Immunologic Non-inferiority of 13-valent Pneumococcal Conjugate Vaccine Compared to 7-valent Pneumococcal Conjugate Vaccine Given as a 4-Dose Series in Healthy Infants and Toddlers, presented at the 48th Annual ICAAC/ISDA 46th Annual Meeting, Washington D.C., Oct. 25-28, 2008.

It is hypothesized that multivalent polysaccharide-protein conjugate vaccines can have reduced immunogenicity if the valency of the vaccine is increased, making it challenging to develop vaccines with high valency. This could be due to multiple mechanisms. Carrier induced epitopic suppression refers to interference with the antibody response to an antigen (such as a capsular polysaccharide) coupled to the same carrier protein. Interference may also arise from competition for a limited number of carrier specific primed T helper cells. As a result, there may be a decrease in response to the shared capsular polysaccharide as the valency of a conjugate vaccine is increased. This observation was noted as the vaccine valency increased from a 7-valent vaccine to 13-valent vaccine (See, Comparison of IgG antibody GMC of Prevnar 7 vs Prevnar 13, table 9, page 29 of PCV13 monograph). Therefore, there is a need to identify methods of treatment for pneumococcal disease that employ vaccines that are effective against many different pneumococci expressing serotypes yet utilize the lowest valency of polysaccharide-protein conjugates in a multivalent vaccine.

### SUMMARY OF THE INVENTION

The present invention provides an immunogenic multivalent pneumococcal polysaccharide-protein conjugate vaccine composition comprising a S. pneumoniae serotype 35B polysaccharide-protein conjugate for use in a method for preventing, treating or ameliorating an infection, disease or condition caused by S. pneumoniae serotype 29 in a subject, wherein said composition does not comprise a S. pneumoniae serotype 29 polysaccharide-protein conjugate.

The invention also provides methods for preventing, treating or ameliorating an infection, disease or condition caused by S. pneumoniae serotype 29 in a subject by administering an immunogenic multivalent pneumococcal polysaccharide-protein conjugate vaccine composition which comprises a serotype 35B polysaccharide-protein conjugate, wherein said vaccine composition does not comprise a serotype 29 polysaccharide-protein conjugate.

### BRIEF DESCRIPTION OF THE DRAWINGS

FIG. 1. OPA titers of sera from rabbits (A) and mice (CD1 mouse (B) and SW mouse (C)) immunized with 35B-CRM197/APA. Pre-immune sera and PD3 mice sera were tested as a pool. PD2 rabbit sera were tested individually. Error bars represent the 95% confidence interval of GMTs.

FIG. 2. OPA titers of sera from rabbits immunized with PCV21. Pre-immune sera were tested as a pool. PD2 rabbit sera were tested individually in anti-35B OPA and as a pool for each group in anti-29 OPA assay. Error bars represent the 95% confidence interval of GMTs.

FIG. 3. ELISA IgG titers of sera from mice immunized with 35B-CRM197 vaccine at PD3. Error bars represent the 95% confidence interval of GMTs.

FIG. 4. OPA titers of sera from mice immunized with 35B-CRM197 vaccine at PD3. Error bars represent the 95% confidence interval of GMTs.

FIG. 5. Survival of serotype 29 IT challenge.

### DETAILED DESCRIPTION OF THE INVENTION

The present invention provides an immunogenic multivalent pneumococcal polysaccharide-protein conjugate composition comprising a S. pneumoniae serotype 35B polysaccharide-protein conjugate for use in a method for preventing, treating or ameliorating an infection, disease or condition caused by S. pneumoniae serotype 29 in a subject, wherein said composition does not comprise a S. pneumoniae serotype 29 polysaccharide-protein conjugate.

### Embodiment 1

In another embodiment the present invention provides the immunogenic composition of Embodiment 1, further comprising polysaccharide-protein conjugates from S. pneumoniae serotypes 4, 6B, 9V, 14, 18C, 19F and 23F.

In another embodiment the present invention provides the immunogenic composition of Embodiment 1, further comprising polysaccharide-protein conjugates from S. pneumoniae serotypes 1, 3, 4, 5, 6A, 6B, 7F, 9V, 14, 18C, 19A, 19F and 23F.

In another embodiment the present invention provides the immunogenic composition of Embodiment 1, further comprising polysaccharide-protein conjugates from S. pneumoniae serotypes 1, 3, 4, 5, 6A, 6B, 7F, 9V, 14, 18C, 19A, 19F, 22F, 23F and 33F.

In another embodiment the present invention provides the immunogenic composition of Embodiment 1, further comprising polysaccharide-protein conjugates from S. pneumoniae serotypes 1, 3, 4, 5, 6A, 6B, 7F, 8, 9V, 10A, 11A, 12F, 14, 15B/C, 18C, 19A, 19F, 22F, 23F and 33F.

In another embodiment the present invention provides the immunogenic composition of Embodiment 1, further comprising polysaccharide-protein conjugates from S. pneumoniae serotypes 8, 10A, 11A, 12F, 15B/C, 22F and 33F.

In an embodiment, the invention provides an immunogenic multivalent pneumococcal polysaccharide-protein conjugate composition comprising S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B for use in a method for preventing, treating or ameliorating an infection, disease or condition caused by S. pneumoniae serotype 29 in a subject, wherein said composition does not comprise a S. pneumoniae serotype 29 polysaccharide-protein conjugate.

In an embodiment, the invention provides an immunogenic multivalent pneumococcal polysaccharide-protein conjugate composition consisting of S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B for use in a method for preventing, treating or ameliorating an infection, disease or condition caused by S. pneumoniae serotype 29 in a subject.

The invention further provides methods for preventing, treating or ameliorating an infection, disease or condition caused by S. pneumoniae serotype 29 in a subject by administering an immunogenic multivalent pneumococcal polysaccharide-protein conjugate vaccine composition which comprises a serotype 35B polysaccharide-protein conjugate, wherein said vaccine composition does not comprise a serotype 29 polysaccharide-protein conjugate.

### Embodiment 2

In an embodiment, the invention also provides the Embodiment 2 method above, wherein the vaccine composition further comprises S. pneumoniae polysaccharide-protein conjugates from serotypes 4, 6B, 9V, 14, 18C, 19F and 23F.

In another embodiment, the invention also provides the Embodiment 2 method above, wherein the vaccine composition further comprises S. pneumoniae polysaccharide-protein conjugates from serotypes 1, 3, 4, 5, 6A, 6B, 7F, 9V, 14, 18C, 19A, 19F and 23F.

In another embodiment, the invention also provides the Embodiment 2 method above, wherein the vaccine composition further comprises S. pneumoniae polysaccharide-protein conjugates from serotypes 1, 3, 4, 5, 6A, 6B, 7F, 9V, 14, 18C, 19A, 19F, 22F, 23F and 33F.

In another embodiment, the invention also provides the Embodiment 2 method above, wherein the vaccine composition further comprises S. pneumoniae polysaccharide-protein conjugates from serotypes 1, 3, 4, 5, 6A, 6B, 7F, 8, 9V, 10A, 11A, 12F, 14, 15B/C, 18C, 19A, 19F, 22F, 23F and 33F.

The invention also provides the Embodiment 2 method, wherein the vaccine composition, as defined in any of the embodiments above, has no more than 10 additional S. pneumoniae serotype polysaccharide-protein conjugates, or has 6 additional S. pneumoniae serotype polysaccharide-protein conjugates, or has 5 additional S. pneumoniae serotype polysaccharide-protein conjugates, or has 4 additional S. pneumoniae serotype polysaccharide-protein conjugates, or has 3 additional S. pneumoniae serotype polysaccharide-protein conjugates, or has 2 additional S. pneumoniae serotype polysaccharide-protein conjugates, or has 1 additional S. pneumoniae serotype polysaccharide-protein conjugate.

In an embodiment, the invention further provides the methods above wherein the 6 additional S. pneumoniae polysaccharide-protein conjugates are from serotypes 16F, 23A, 31, 23B, 24F, and 15A.

In an embodiment, the invention further provides the methods above wherein the 4 additional S. pneumoniae polysaccharide-protein conjugates are from serotypes 2, 9N, 17F and 20.

In an embodiment, the invention further provides the methods above wherein the 3 additional S. pneumoniae polysaccharide-protein conjugate are from serotypes 23B, 24F and 15A.

In another embodiment, the invention further provides the methods above wherein the 2 additional S. pneumoniae polysaccharide-protein conjugates are from serotypes 23B and 15A.

In another embodiment, the invention further provides the methods above wherein the 1 additional S. pneumoniae polysaccharide-protein conjugate is from serotype 9N.

In another embodiment, the invention further provides the methods above wherein the 1 additional S. pneumoniae polysaccharide-protein conjugate is from serotype 17F.

In another embodiment, the invention further provides the methods above wherein the 1 additional S. pneumoniae polysaccharide-protein conjugate is from serotype 20.

In another embodiment, the invention further provides the methods above wherein the 1 additional S. pneumoniae polysaccharide-protein conjugate is from serotype 23B.

In another embodiment, the invention further provides the methods above wherein the 1 additional S. pneumoniae polysaccharide-protein conjugate is from serotype 15A.

In an embodiment, the invention further provides methods for preventing, treating or ameliorating an infection, disease or condition caused by S. pneumoniae serotype 29 in a subject by administering an immunogenic multivalent pneumococcal polysaccharide-protein conjugate vaccine composition which comprises a serotype 35B polysaccharide-protein conjugate, wherein said vaccine does not comprise a serotype 29 polysaccharide-protein conjugate (Embodiment 3).

In another aspect, the invention also provides the Embodiment 3 method above, wherein the vaccine composition comprises S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B.

In another aspect, the invention also provides the Embodiment 3 method above, wherein the vaccine composition consists of S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B.

The present invention provides an immunogenic multivalent pneumococcal polysaccharide-protein conjugate composition comprising a S. pneumoniae serotype 29 polysaccharide-protein conjugate for use in a method for preventing, treating or ameliorating an infection, disease or condition caused by S. pneumoniae serotype 35B in a subject, wherein said composition does not comprise a S. pneumoniae serotype 35B polysaccharide-protein conjugate (Embodiment 4)

In an embodiment, the present invention provides the immunogenic composition of Embodiment 4 further comprising S. pneumoniae polysaccharide-protein conjugates from serotypes 4, 6B, 9V, 14, 18C, 19F and 23F.

In another embodiment, the present invention provides the immunogenic composition of Embodiment 4 further comprising S. pneumoniae polysaccharide-protein conjugates from serotypes 1, 3, 4, 5, 6A, 6B, 7F, 9V, 14, 18C, 19A, 19F and 23F.

In another embodiment, the present invention provides the immunogenic composition of Embodiment 4 further comprising S. pneumoniae polysaccharide-protein conjugates from serotypes 1, 3, 4, 5, 6A, 6B, 7F, 9V, 14, 18C, 19A, 19F, 22F, 23F and 33F.

In another embodiment, the present invention provides the immunogenic composition of Embodiment 4 further comprising S. pneumoniae polysaccharide-protein conjugates from serotypes 1, 3, 4, 5, 6A, 6B, 7F, 8, 9V, 10A, 11A, 12F, 14, 15B/C, 18C, 19A, 19F, 22F, 23F and 33F.

In another embodiment, the present invention provides the immunogenic composition of Embodiment 4 further comprising S. pneumoniae polysaccharide-protein conjugates from serotypes 8, 10A, 11A, 12F, 15B/C, 22F and 33F.

The invention further provides methods for preventing, treating or ameliorating an infection, disease or condition caused by S. pneumoniae serotype 35B in a subject by administering an immunogenic multivalent pneumococcal polysaccharide-protein conjugate vaccine composition which comprises a serotype 29 polysaccharide-protein conjugate, wherein said vaccine composition does not comprise a serotype 35B polysaccharide-protein conjugate.

### Embodiment 5

In an embodiment, the invention also provides the method above of Embodiment 5, wherein the vaccine composition further comprises S. pneumoniae polysaccharide-protein conjugates from serotypes 4, 6B, 9V, 14, 18C, 19F and 23F.

In another embodiment the invention also provides the method above of Embodiment 5, wherein the vaccine composition further comprises S. pneumoniae polysaccharide-protein conjugates from serotypes 1, 3, 4, 5, 6A, 6B, 7F, 9V, 14, 18C, 19A, 19F and 23F.

In another embodiment, the invention also provides the method above of Embodiment 5, wherein the vaccine composition further comprises S. pneumoniae polysaccharide-protein conjugates from serotypes 1, 3, 4, 5, 6A, 6B, 7F, 9V, 14, 18C, 19A, 19F, 22F, 23F and 33F.

In another embodiment, the invention also provides the method above of Embodiment 5, wherein the vaccine composition further comprises S. pneumoniae polysaccharide-protein conjugates from serotypes 1, 3, 4, 5, 6A, 6B, 7F, 8, 9V, 10A, 11A, 12F, 14, 15B/C, 18C, 19A, 19F, 22F, 23F and 33F.

### Definitions and Abbreviations

As used throughout the specification and appended claims, the following abbreviations apply:
APA aluminum phosphate adjuvant
CI confidence interval
DMSO dimethyl sulfoxide
DS polysaccharide-protein Drug Substance
GMT geometric mean titer
HPSEC high performance size exclusion chromatography
IM intra-muscular or intra-muscularly
LOS lipo-oligosaccharide
LPS lipopolysaccharide
MALS multi-angle light scattering
MBC monovalent bulk conjugate
Mn number averaged molecular weight
MOPA multiplexed opsonophagocytosis assays
MW molecular weight
NMWCO nominal molecular weight cut off
NZWR New Zealand White rabbit
OPA opsonophagocytosis assay
PCV pneumococcal conjugate vaccine
PD1 post-dose 1
PD2 post-dose 2
PD3 post-dose 3
PnPs Pneumococcal Polysaccharide
Ps polysaccharide
PS-20 polysorbate-20
RI refractive index
UV ultraviolet
w/v weight per volume

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

As used herein, the term “comprises” when used with the immunogenic composition of the invention refers to the inclusion of any other components (subject to limitations of “consisting of” language for the antigen mixture), such as adjuvants and excipients. The term “consisting of” when used with the multivalent polysaccharide-protein conjugate mixture refers to a mixture having those particular S. pneumoniae polysaccharide protein conjugates and no other S. pneumoniae polysaccharide protein conjugates from a different serotype.

“Effective amount” of a composition and/or vaccine of the invention refers to a dose required to elicit antibodies that significantly reduce the likelihood or severity of infectivity of a microbe, e.g., S. pneumoniae, during a subsequent challenge.

As used herein, the phrase “indicated for the prevention of pneumococcal disease” means that a vaccine or immunogenic composition is approved by one or more regulatory authorities, such as the US Food and Drug Administration, for the prophylaxis of one or more diseases caused by any serotype of S. pneumoniae, including, but not limited to: pneumococcal disease generally, pneumococcal pneumonia, pneumococcal meningitis, pneumococcal bacteremia, invasive disease caused by S. pneumoniae, and otitis media caused by S. pneumoniae.

An “immunogenic multivalent pneumococcal polysaccharide-protein conjugate vaccine composition” is a pharmaceutical preparation comprising more than one active agent (e.g., pneumococcal polysaccharide-protein conjugate) that provides active immunity to disease or pathological condition caused by more than one serotype of S. pneumoniae.

An “adjuvant,” as defined herein, is a substance that serves to enhance the immunogenicity of an immunogenic composition and/or vaccine of the invention. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan.

A “patient” (alternatively referred to herein as a “subject”) refers to a mammal capable of being infected with a S. pneumoniae. In preferred embodiments, the patient is a human. A patient can be treated prophylactically or therapeutically. Prophylactic treatment provides sufficient protective immunity to reduce the likelihood or severity of a pneumococcal infection or the effects thereof, e.g., pneumococcal pneumonia. Therapeutic treatment can be performed to reduce the severity or prevent recurrence of a S. pneumoniae infection or the clinical effects thereof. Prophylactic treatment can be performed using a multivalent immunogenic composition of the invention, as described herein. The composition of the invention can be administered to the general population or to those persons at an increased risk of pneumococcal infection, e.g. infants, children and the elderly, or those who live with or care for the elderly. As disclosed herein, the immunogenic compositions described herein may be used in various therapeutic or prophylactic methods for preventing, treating or ameliorating a bacterial infection, disease or condition in a subject.
The term “15B/C” refers to serotype 15B and/or serotype 15C.

### General Methods for Making Multivalent Pneumococcal Polysaccharide-Protein Conjugate Vaccines

### Capsular Polysaccharides

Bacterial capsular polysaccharides, particularly those that have been used as antigens, are suitable for use in the invention and can readily be identified by methods for identifying immunogenic and/or antigenic polysaccharides. Example bacterial capsular polysaccharides from S. pneumoniae are serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38.

Polysaccharides can be purified by known techniques. The invention is not limited to polysaccharides purified from natural sources, however, and the polysaccharides may be obtained by other methods, such as total or partial synthesis. Capsular polysaccharides from S. pneumoniae can be prepared by standard techniques known to those skilled in the art. For example, polysaccharides can be isolated from bacteria and may be sized to some degree by known methods (see, e.g., European Patent Nos. EP497524 and EP497525); and preferably by microfluidization accomplished using a homogenizer or by chemical hydrolysis. S. pneumoniae strains corresponding to each polysaccharide serotype may be grown in a soy-based medium. The individual polysaccharides may then be purified through standard steps including centrifugation, precipitation, and ultrafiltration. See, e.g., U.S. Patent Application Publication No. 2008/0286838 and U.S. Pat. No. 5,847,112. Polysaccharides can be sized in order to reduce viscosity and/or to improve filterability and the lot-to-lot consistency of subsequent conjugated products.

Purified polysaccharides can be chemically activated to introduce functionalities capable of reacting with a carrier protein using standard techniques. Chemical activation of polysaccharides and subsequent conjugation to carrier protein(s) are achieved by means described in U.S. Pat. Nos. 4,365,170, 4,673,574 and 4,902,506. Briefly, the pneumococcal polysaccharide is reacted with a periodate-based oxidizing agent such as sodium periodate, potassium periodate, or periodic acid resulting in oxidative cleavage of vicinal hydroxyl groups to generate reactive aldehyde groups. Suitable molar equivalents of periodate (e.g., sodium periodate, sodium metaperiodate and the like) include 0.05 to 0.5 molar equivalents (molar ratio of periodate to polysaccharide repeat unit) or 0.1 to 0.5 molar equivalents. The periodate reaction can be varied from 30 minutes to 24 hours depending on the diol conformation (e.g., acyclic diols, cis diols, trans diols), which controls accessibility of the reactive hydroxyl groups to the sodium periodate.

The term “periodate” includes both periodate and periodic acid; the term also includes both metaperiodate (IO4-) and orthoperiodate (IO6-) and includes the various salts of periodate (e.g., sodium periodate and potassium periodate). The capsular polysaccharide may be oxidized in the presence of metaperiodate, or in the presence of sodium periodate (NaIO4). Further, the capsular polysaccharide may be oxidized in the presence of orthoperiodate, or in the presence of periodic acid.

Purified polysaccharides can also be connected to a linker. Once activated or connected to a linker, each capsular polysaccharide is separately conjugated to a carrier protein to form a glycoconjugate. The polysaccharide conjugates may be prepared by known coupling techniques.

The polysaccharide can be coupled to a linker to form a polysaccharide-linker intermediate in which the free terminus of the linker is an ester group. The linker is therefore one in which at least one terminus is an ester group. The other terminus is selected so that it can react with the polysaccharide to form the polysaccharide-linker intermediate.

The polysaccharide can be coupled to a linker using a primary amine group in the polysaccharide. In this case, the linker typically has an ester group at both termini. This allows the coupling to take place by reacting one of the ester groups with the primary amine group in the polysaccharide by nucleophilic acyl substitution. The reaction results in a polysaccharide-linker intermediate in which the polysaccharide is coupled to the linker via an amide linkage. The linker is therefore a bifunctional linker that provides a first ester group for reacting with the primary amine group in the polysaccharide and a second ester group for reacting with the primary amine group in the carrier molecule. A typical linker is adipic acid N-hydroxysuccinimide diester (SIDEA).

The coupling can also take place indirectly, i.e. with an additional linker that is used to derivatize the polysaccharide prior to coupling to the linker.

The polysaccharide can be coupled to the additional linker using a carbonyl group at the reducing terminus of the polysaccharide. This coupling comprises two steps: (a1) reacting the carbonyl group with the additional linker; and (a2) reacting the free terminus of the additional linker with the linker. In these embodiments, the additional linker typically has a primary amine group at both termini, thereby allowing step (a1) to take place by reacting one of the primary amine groups with the carbonyl group in the polysaccharide by reductive amination. A primary amine group is used that is reactive with the carbonyl group in the polysaccharide. Hydrazide or hydroxylamino groups are suitable. The same primary amine group is typically present at both termini of the additional linker which allows for the possibility of polysaccharide (Ps)-Ps coupling. The reaction results in a polysaccharide-additional linker intermediate in which the polysaccharide is coupled to the additional linker via a C—N linkage.

The polysaccharide can be coupled to the additional linker using a different group in the polysaccharide, particularly a carboxyl group. This coupling comprises two steps: (a1) reacting the group with the additional linker; and (a2) reacting the free terminus of the additional linker with the linker. In this case, the additional linker typically has a primary amine group at both termini, thereby allowing step (a1) to take place by reacting one of the primary amine groups with the carboxyl group in the polysaccharide by EDAC activation. A primary amine group is used that is reactive with the EDAC-activated carboxyl group in the polysaccharide. A hydrazide group is suitable. The same primary amine group is typically present at both termini of the additional linker. The reaction results in a polysaccharide-additional linker intermediate in which the polysaccharide is coupled to the additional linker via an amide linkage.

### Carrier Protein

CRM197 is preferably used as the carrier protein. CRM197 is a non-toxic variant (i.e., toxoid) of diphtheria toxin. CRM197 may be isolated from cultures of Corynebacterium diphtheria strain C7 (β197) grown in casamino acids and yeast extract-based medium. CRM197 may be prepared recombinantly in accordance with the methods described in U.S. Pat. No. 5,614,382. Typically, CRM197 is purified through a combination of ultrafiltration, ammonium sulfate precipitation, and ion-exchange chromatography. Further CRM197 may be prepared in Pseudomonas fluorescens using Pfenex Expression Technology™ (Pfenex Inc., San Diego, Calif.).

Other suitable carrier proteins include additional inactivated bacterial toxins such as DT (Diphtheria toxoid), TT (tetanus toxoid) or fragment C of TT, pertussis toxoid, cholera toxoid (e.g., as described in International Patent Application Publication No. WO 2004/083251), E. coli LT, E. coli ST, and exotoxin A from Pseudomonas aeruginosa. Bacterial outer membrane proteins such as outer membrane complex c (OMPC), porins, transferrin binding proteins, pneumococcal surface protein A (PspA; See International Application Patent Publication No. WO 02/091998), pneumococcal surface adhesin protein (PsaA), C5a peptidase from Group A or Group B streptococcus, or Haemophilus influenzae protein D, pneumococcal pneumolysin (Kuo et al., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; see, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Patent Application Publication No. WO 01/72337), toxin A or B of C. difficile (See International Patent Publication No. WO 00/61761), and flagellin (See Ben-Yedidia et al., 1998, Immunol Lett 64:9) can also be used as carrier proteins.

Where multivalent vaccines are used, a second carrier can be used for one or more of the antigens in a multivalent vaccine. The second carrier protein is preferably a protein that is non-toxic and non-reactogenic and obtainable in sufficient amount and purity. The second carrier protein is also conjugated or joined with an antigen, e.g., a S. pneumoniae polysaccharide to enhance immunogenicity of the antigen. Carrier proteins should be amenable to standard conjugation procedures. Each capsular polysaccharide not conjugated to a first carrier protein may be conjugated to the same second carrier protein (e.g., each capsular polysaccharide molecule being conjugated to a single carrier protein). Capsular polysaccharides not conjugated to a first carrier protein may be conjugated to two or more carrier proteins (each capsular polysaccharide molecule being conjugated to a single carrier protein). In such embodiments, each capsular polysaccharide of the same serotype is typically conjugated to the same carrier protein. Other DT mutants can be used as the second carrier protein, such as CRM176, CRM228, CRM45 (Uchida et al., 1973, J Biol Chem 218:3838-3844); CRM9, CRM45 CRM102, CRM103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or 4,950,740; mutation of at least one or more residues Lys 516, Lys 526, Phe 530 and/or Lys 534 and other mutations disclosed in U.S. Pat. No. 5,917,017 or 6,455,673; or fragment disclosed in U.S. Pat. No. 5,843,711.

### Conjugation by Reductive Amination

Covalent coupling of polysaccharide to carrier protein can be performed via reductive amination in which an amine-reactive moiety on the polysaccharide is directly coupled to primary amine groups (mainly lysine residues) of the protein. As is well known, a reductive amination reaction proceeds via a two step mechanism. First, a Schiff base intermediate, of formula R—CH═N—R′, is formed by reaction of an aldehyde group on molecule 1 (R—CHO) with a primary amine group (R′—NH2) on molecule 2. In the second step, the Schiff base is reduced to form an amino compound of formula R—CH2-NH—R′. While many reducing agents are capable of being utilized, most often a highly selective reducing agent such as sodium cyanoborohydride (NaCNBH3) is employed since such reagents will specifically reduce only the imine function of the Schiff base.

Since all the polysaccharides have an aldehyde function at the end of the chain (terminal aldehyde function), the conjugation methods comprising a reductive amination of the polysaccharide can be applied very generally and, when there is no other aldehyde function in the repeating unit (intrachain aldehyde function), such methods make it possible to obtain conjugates in which a polysaccharide molecule is coupled to a single molecule of carrier protein.

A typical reducing agent is cyanoborohydride salt such as sodium cyanoborohydride. The imine-selective reducing agent typically employed is sodium cyanoborohydride, although other cyanoborohydride salts can be used including potassium cyanoborohydride. Differences in starting cyanide levels in sodium cyanoborohydride reagent lots and residual cyanide in the conjugation reaction can lead to inconsistent conjugation performance, resulting in variable product attributes, such as conjugate size and conjugate Ps-to-CRM197 ratio. By controlling and/or reducing the free cyanide levels in the final reaction product, conjugation variability can be reduced.

Residual unreacted aldehydes on the polysaccharide are optionally reduced with the addition of a strong reducing agent, such as sodium borohydride. Generally, use of a strong reducing agent is preferred. However, for some polysaccharides, it is preferred to avoid this step. For example, S. pneumoniae serotype 5 contains a ketone group that may react readily with a strong reductant. In this case, it is preferable to bypass the reduction step to protect the antigenic structure of the polysaccharide.

Following conjugation, the polysaccharide-protein conjugates may be purified to remove excess conjugation reagents as well as residual free protein and free polysaccharide by one or more of any techniques well known to the skilled artisan, including concentration/diafiltration operations, ultrafiltration, precipitation/elution, column chromatography, and depth filtration. See, e.g., U.S. Pat. No. 6,146,902. The purifying step may be handled by ultrafiltration.

### Multivalent Polysaccharide-Protein Conjugate Vaccines

Immunogenic compositions can comprise capsular polysaccharides from S. pneumoniae serotypes selected from at least one of 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A or 20B), 22F, 23A, 23B, 23F, 24F, 33F, 35B, 35F, or 38 conjugated to one or more carrier proteins. Preferably, saccharides from a particular serotype are not conjugated to more than one carrier protein.

After the individual glycoconjugates are purified, they may be compounded to formulate immunogenic compositions of the present invention. These pneumococcal conjugates are prepared by separate processes and bulk formulated into a single dosage formulation.

### Pharmaceutical/Vaccine Compositions

The present invention further provides compositions, including pharmaceutical, immunogenic and vaccine compositions, comprising, consisting essentially of, or alternatively, consisting of any of the polysaccharide serotype combinations described above together with a pharmaceutically acceptable carrier and an adjuvant. In one embodiment, the compositions comprise, consist essentially of, or consist of 2 to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 distinct polysaccharide-protein conjugates, wherein each of the conjugates contains a different capsular polysaccharide conjugated to either the first carrier protein or the second carrier protein, and wherein the capsular polysaccharides from at least one of serotypes 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A or 20B), 22F, 23A, 23B, 23F, 24F, 33F, 35B, 35F, or 38 of Streptococcus pneumoniae are conjugated to CRM197.

The present invention provides an immunogenic multivalent pneumococcal polysaccharide-protein conjugate composition comprising a S. pneumoniae serotype 35B polysaccharide-protein conjugate for use in a method for preventing, treating or ameliorating an infection, disease or condition caused by S. pneumoniae serotype 29 in a subject, wherein said composition does not comprise a S. pneumoniae serotype 29 polysaccharide-protein conjugate.

### Embodiment 1

In another embodiment, the present invention provides the immunogenic composition of Embodiment 1, further comprising S. pneumoniae polysaccharide-protein conjugates from serotypes 4, 6B, 9V, 14, 18C, 19F and 23F.

In another embodiment, the present invention provides the immunogenic composition of Embodiment 1, further comprising S. pneumoniae polysaccharide-protein conjugates from serotypes 1, 3, 4, 5, 6A, 6B, 7F, 9V, 14, 18C, 19A, 19F and 23F.

In another embodiment, the present invention provides the immunogenic composition of Embodiment 1, further comprising S. pneumoniae polysaccharide-protein conjugates from serotypes 1, 3, 4, 5, 6A, 6B, 7F, 9V, 14, 18C, 19A, 19F, 22F, 23F and 33F.

In another embodiment, the present invention provides the immunogenic composition of Embodiment 1, further comprising S. pneumoniae polysaccharide-protein conjugates from serotypes 1, 3, 4, 5, 6A, 6B, 7F, 8, 9V, 10A, 11A, 12F, 14, 15B/C, 18C, 19A, 19F, 22F, 23F and 33F.

In another embodiment, the present invention provides the immunogenic composition of Embodiment 1, further comprising S. pneumoniae polysaccharide-protein conjugates from serotypes 8, 10A, 11A, 12F, 15B/C, 22F and 33F.

The present invention provides an immunogenic composition, as defined in any of the compositions above, for use in a method for preventing, treating or ameliorating an infection, disease or condition caused by S. pneumoniae serotype 29 in a subject, wherein the composition comprises a S. pneumoniae serotype 35B polysaccharide-protein conjugate but does not comprise a S. pneumoniae serotype 29 polysaccharide-protein conjugate, and wherein the composition has no more than 10 additional S. pneumoniae serotype polysaccharide-protein conjugates. As used herein, “additional S. pneumoniae serotype polysaccharide-protein conjugates” refers to S. pneumoniae polysaccharide-protein conjugates other than from serotype 35B.

In a further embodiment, the composition has 6 additional S. pneumoniae serotype polysaccharide-protein conjugates, preferably wherein the 6 additional S. pneumoniae serotype polysaccharide-protein conjugates are from serotypes 16F, 23A, 31, 23B, 24F and 15A.

In another embodiment, the composition has 5 additional S. pneumoniae serotype polysaccharide-protein conjugates.

In another embodiment, the composition has no more than 4 additional S. pneumoniae serotype polysaccharide-protein conjugates, preferably wherein the no more than 4 additional S. pneumoniae serotype polysaccharide-protein conjugates are selected from serotypes 2, 9N, 17F and 20.

In another embodiment, the composition has 3 additional S. pneumoniae serotype polysaccharide-protein conjugates, preferably wherein the 3 additional S. pneumoniae serotype polysaccharide-protein conjugates are from serotypes 23B, 24F and 15A.

In another embodiment, the composition has 2 additional S. pneumoniae serotype polysaccharide-protein conjugates, preferably wherein the 2 additional S. pneumoniae serotype polysaccharide-protein conjugates are from serotypes 23B and 15A.

In another embodiment, wherein the composition has 1 additional S. pneumoniae serotype polysaccharide-protein conjugate, preferably wherein the 1 additional S. pneumoniae serotype polysaccharide-protein conjugate is from serotype 9N, or serotype 17F, or serotype 20, or serotype 20A, or serotype 20B, or serotype 23B, or serotype 15A.

In an embodiment the invention provides an immunogenic multivalent pneumococcal polysaccharide-protein conjugate composition comprising S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B for use in a method for preventing, treating or ameliorating an infection, disease or condition caused by S. pneumoniae serotype 29 in a subject, wherein said composition does not comprise a S. pneumoniae serotype 29 polysaccharide-protein conjugate.

In an embodiment, the invention provides an immunogenic multivalent pneumococcal polysaccharide-protein conjugate composition consisting of S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B for use in a method for preventing, treating or ameliorating an infection, disease or condition caused by S. pneumoniae serotype 29 in a subject.

Formulation of the S. pneumoniae polysaccharide-protein conjugates of the present invention can be accomplished using art-recognized methods. For instance, individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

In a preferred embodiment, the vaccine composition is formulated in L-histidine buffer with sodium chloride.

As defined herein, an “adjuvant” is a substance that serves to enhance the immunogenicity of an immunogenic composition of the invention. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan. Suitable adjuvants to enhance effectiveness of the composition include, but are not limited to:

(1) aluminum salts (alum), such as aluminum hydroxide, aluminum phosphate, aluminum sulfate, etc.;

(2) oil-in-water emulsion formulations (with or without other specific immunostimulating agents such as muramyl peptides (defined below) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP either microfluidized into a submicron emulsion or vortexed to generate a larger particle size emulsion, (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094, trehalose dimycolate (TDM), and cell wall skeleton (CWS), preferably MPL+CWS (Detox™); and (d) a Montanide ISA;

(3) saponin adjuvants, such as Quil A or STIMULON™ QS-21 (Antigenics, Framingham, Mass.) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix® (having essentially the same structure as an ISCOM but without the protein);

(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetradecanoylamino]ethyl 2-Deoxy-4-O-phosphono-3-O—[(R)-3-tetradecanoyloxytetradecanoyl]-2-[(R)-3-tetradecanoyloxytetradecanoylamino]-b-D-glucopyranoside, which is also known as 529 (formerly known as RC529), which is formulated as an aqueous form or as a stable emulsion;

(5) synthetic polynucleotides such as oligonucleotides containing CpG motif(s) (U.S. Pat. No. 6,207,646);

(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.; and

(7) complement, such as a trimer of complement component C3d.

In another embodiment, the adjuvant is a mixture of 2, 3, or more of the above adjuvants, e.g., SBAS2 (an oil-in-water emulsion also containing 3-deacylated monophosphoryl lipid A and QS21).

Muramyl peptides include, but are not limited to, N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE), etc.

In certain embodiments, the adjuvant is an aluminum salt. The aluminum salt adjuvant may be an alum-precipitated vaccine or an alum-adsorbed vaccine. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, alhydrogel, Superfos, Amphogel, aluminum (III) hydroxide, aluminum hydroxyphosphate sulfate (Aluminum Phosphate Adjuvant (APA)), amorphous alumina, trihydrated alumina, or trihydroxyaluminum.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and sterilized (either steam sterilization or autoclaving).

In certain embodiments, a commercially available Al(OH)₃ (e.g. Alhydrogel or Superfos of Denmark/Accurate Chemical and Scientific Co., Westbury, N.Y.) is used to adsorb proteins. Adsorption of protein is dependent, in another embodiment, on the pI (Isoelectric pH) of the protein and the pH of the medium. A protein with a lower pI adsorbs to the positively charged aluminum ion more strongly than a protein with a higher pI. Aluminum salts may establish a depot of antigen that is released slowly over a period of 2-3 weeks, be involved in nonspecific activation of macrophages and complement activation, and/or stimulate innate immune mechanism (possibly through stimulation of uric acid). See, e.g., Lambrecht et al., 2009, Curr Opin Immunol 21:23.

Monovalent bulk aqueous conjugates are typically blended together and diluted to target 8 μg/mL for all serotypes except 6B, which will be diluted to target 16 μg/mL. Once diluted, the batch will be filter sterilized, and an equal volume of aluminum phosphate adjuvant added aseptically to target a final aluminum concentration of 250 μg/mL. The adjuvanted, formulated batch will be filled into single-use, 0.5 mL/dose vials.

In certain embodiments, the adjuvant is a CpG-containing nucleotide sequence, for example, a CpG-containing oligonucleotide, in particular, a CpG-containing oligodeoxynucleotide (CpG ODN). In another embodiment, the adjuvant is ODN 1826, which may be acquired from Coley Pharmaceutical Group.

“CpG-containing nucleotide,” “CpG-containing oligonucleotide,” “CpG oligonucleotide,” and similar terms refer to a nucleotide molecule of 6-50 nucleotides in length that contains an unmethylated CpG moiety. See, e.g., Wang et al., 2003, Vaccine 21:4297. In another embodiment, any other art-accepted definition of the terms is intended. CpG-containing oligonucleotides include modified oligonucleotides using any synthetic internucleoside linkages, modified base and/or modified sugar.

Methods for use of CpG oligonucleotides are well known in the art and are described, for example, in Sur et al., 1999, J Immunol. 162:6284-93; Verthelyi, 2006, Methods Mol Med. 127:139-58; and Yasuda et al., 2006, Crit Rev Ther Drug Carrier Syst. 23:89-110.

### Administration/Dosage

The compositions and formulations of the present invention can be used to protect or treat a human susceptible to infection, e.g., a pneumococcal infection, by means of administering the vaccine via a systemic or mucosal route. In one embodiment, the present invention provides a method of inducing an immune response to a S. pneumoniae capsular polysaccharide conjugate, comprising administering to a human an immunologically effective amount of an immunogenic composition of the present invention. In another embodiment, the present invention provides a method of vaccinating a human against a pneumococcal infection, comprising the step of administering to the human an immunologically effective amount of an immunogenic composition of the present invention.

Optimal amounts of components for a particular vaccine can be ascertained by standard studies involving observation of appropriate immune responses in subjects. For example, in another embodiment, the dosage for human vaccination is determined by extrapolation from animal studies to human data. In another embodiment, the dosage is determined empirically.

“Effective amount” of a composition of the invention refers to a dose required to elicit antibodies that significantly reduce the likelihood or severity of infectivity of a microbe, e.g., S. pneumonia, during a subsequent challenge.

The methods of the invention can be used for the prevention and/or reduction of primary clinical syndromes caused by microbes, e.g., S. pneumonia, including both invasive infections (meningitis, pneumonia, and bacteremia), and noninvasive infections (acute otitis media, and sinusitis).

Administration of the compositions of the invention can include one or more of: injection via the intramuscular, intraperitoneal, intradermal or subcutaneous routes; or via mucosal administration to the oral/alimentary, respiratory or genitourinary tracts. In one embodiment, intranasal administration is used for the treatment of pneumonia or otitis media (as nasopharyngeal carriage of pneumococci can be more effectively prevented, thus attenuating infection at its earliest stage).

The amount of conjugate in each vaccine dose is selected as an amount that induces an immunoprotective response without significant, adverse effects. Such amount can vary depending upon the pneumococcal serotype. Generally, for polysaccharide-based conjugates, each dose will comprise 0.1 to 100 μg of each polysaccharide, particularly 0.1 to 10 μg, and more particularly 1 to 5 μg. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 μg.

In one embodiment, the dose of the aluminum salt is 10, 15, 20, 25, 30, 50, 70, 100, 125, 150, 200, 300, 500, or 700 μg, or 1, 1.2, 1.5, 2, 3, 5 mg or more. In yet another embodiment, the dose of alum salt described above is per μg of recombinant protein.

According to any of the methods of the present invention and in one embodiment, the subject is human. In certain embodiments, the human patient is an infant (less than 1 year of age), toddler (approximately 12 to 24 months), or young child (approximately 2 to 5 years). In other embodiments, the human patient is an elderly patient (>65 years). The compositions of this invention are also suitable for use with older children, adolescents and adults (e.g., aged 18 to 45 years or 18 to 65 years).

The invention further provides methods for preventing, treating or ameliorating an infection, disease or condition caused by S. pneumoniae serotype 29 in a subject by administering an immunogenic multivalent pneumococcal polysaccharide-protein conjugate vaccine composition which comprises a S. pneumoniae serotype 35B polysaccharide-protein conjugate, wherein said vaccine composition does not comprise a S. pneumoniae serotype 29 polysaccharide-protein conjugate. (Embodiment 2)

In an embodiment, the invention also provides the Embodiment 2 method above, wherein the vaccine composition further comprises S. pneumoniae polysaccharide-protein conjugates from serotypes 4, 6B, 9V, 14, 18C, 19F and 23F.

In another embodiment, the invention also provides the Embodiment 2 method above, wherein the vaccine composition further comprises S. pneumoniae polysaccharide-protein conjugates from serotypes 1, 3, 4, 5, 6A, 6B, 7F, 9V, 14, 18C, 19A, 19F and 23F.

In another embodiment, the invention also provides the Embodiment 2 method above, wherein the vaccine composition further comprises S. pneumoniae polysaccharide-protein conjugates from serotypes 1, 3, 4, 5, 6A, 6B, 7F, 9V, 14, 18C, 19A, 19F, 22F, 23F and 33F.

In another embodiment, the invention also provides the Embodiment 2 method above, wherein the vaccine composition further comprises S. pneumoniae polysaccharide-protein conjugates from serotypes 1, 3, 4, 5, 6A, 6B, 7F, 8, 9V, 10A, 11A, 12F, 14, 15B/C, 18C, 19A, 19F, 22F, 23F and 33F.

The invention also provides the Embodiment 2 method, wherein the vaccine composition, as defined in any of the embodiments above, has no more than 10 additional serotype polysaccharide-protein conjugates, or has 6 additional serotype polysaccharide-protein conjugates, or has 5 additional serotype polysaccharide-protein conjugates, or has 4 additional serotype polysaccharide-protein conjugates, or has 3 additional serotype polysaccharide-protein conjugates, or has 2 additional serotype polysaccharide-protein conjugates, or has 1 additional serotype polysaccharide-protein conjugate.

In an embodiment, the invention further provides the methods above wherein the 6 additional S. pneumoniae polysaccharide-protein conjugates are from serotypes 16F, 23A, 31, 23B, 24F, 15A.

In an embodiment, the invention further provides the methods above wherein the 4 additional S. pneumoniae polysaccharide-protein conjugates are from serotypes 2, 9N, 17F and 20.

In an embodiment, the invention further provides the methods above wherein the 3 additional S. pneumoniae polysaccharide-protein conjugates are from serotypes 23B, 24F and 15A.

In another embodiment, the invention further provides the methods above wherein the 2 additional S. pneumoniae polysaccharide-protein conjugates are from serotypes 23B and 15A.

In another embodiment, the invention further provides the methods above wherein the 1 additional S. pneumoniae polysaccharide-protein conjugates is from serotype 9N.

In another embodiment, the invention further provides the methods above wherein the 1 additional S. pneumoniae polysaccharide-protein conjugates is from serotype 17F.

In another embodiment, the invention further provides the methods above wherein the 1 additional S. pneumoniae polysaccharide-protein conjugates is from serotype 20.

In another embodiment, the invention further provides the methods above wherein the 1 additional S. pneumoniae polysaccharide-protein conjugates is from serotype 23B.

In another embodiment the invention further provides the methods above wherein the 1 additional S. pneumoniae polysaccharide-protein conjugates is from serotype 15A.

In an embodiment, the invention further provides methods for preventing, treating or ameliorating an infection, disease or condition caused by S. pneumoniae serotype 29 in a subject by administering an immunogenic multivalent pneumococcal polysaccharide-protein conjugate vaccine composition which comprises a S. pneumoniae serotype 35B polysaccharide-protein conjugate, wherein said vaccine composition does not comprise a S. pneumoniae serotype 29 polysaccharide-protein conjugate (Embodiment 3).

In another aspect, the invention also provides the Embodiment 3 method above, wherein the vaccine composition comprises S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B.

In another aspect, the invention also provides the Embodiment 3 method above, wherein the vaccine composition consists of S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B.

The present invention provides an immunogenic multivalent pneumococcal polysaccharide-protein conjugate composition comprising a S. pneumoniae serotype 29 polysaccharide-protein conjugate for use in a method for preventing, treating or ameliorating an infection, disease or condition caused by S. pneumoniae serotype 35B in a subject, wherein said composition does not comprise a S. pneumoniae serotype 35B polysaccharide-protein conjugate (Embodiment 4)

In an embodiment, the present invention provides the immunogenic composition of Embodiment 4 further comprising S. pneumoniae polysaccharide-protein conjugates from serotypes 4, 6B, 9V, 14, 18C, 19F and 23F.

In another embodiment, the present invention provides the immunogenic composition of Embodiment 4 further comprising S. pneumoniae polysaccharide-protein conjugates from serotypes 1, 3, 4, 5, 6A, 6B, 7F, 9V, 14, 18C, 19A, 19F and 23F.

In another embodiment, the present invention provides the immunogenic composition of Embodiment 4 further comprising S. pneumoniae polysaccharide-protein conjugates from serotypes 1, 3, 4, 5, 6A, 6B, 7F, 9V, 14, 18C, 19A, 19F, 22F, 23F and 33F.

In another embodiment, the present invention provides the immunogenic composition of Embodiment 4 further comprising S. pneumoniae polysaccharide-protein conjugates from serotypes 1, 3, 4, 5, 6A, 6B, 7F, 8, 9V, 10A, 11A, 12F, 14, 15B/C, 18C, 19A, 19F, 22F, 23F and 33F.

In another embodiment, the present invention provides the immunogenic composition of Embodiment 4 further comprising S. pneumoniae polysaccharide-protein conjugates from serotypes 8, 10A, 11A, 12F, 15B/C, 22F and 33F.

The invention further provides methods for preventing, treating or ameliorating an infection, disease or condition caused by S. pneumoniae serotype 35B in a subject by administering an immunogenic multivalent pneumococcal polysaccharide-protein conjugate vaccine which comprises a S. pneumoniae serotype 29 polysaccharide-protein conjugate, wherein said vaccine does not comprise a S. pneumoniae serotype 35B polysaccharide-protein conjugate. (Embodiment 5)

In an embodiment, the invention also provides the method above of Embodiment 5, wherein the vaccine composition further comprises S. pneumoniae polysaccharide-protein conjugates from serotypes 4, 6B, 9V, 14, 18C, 19F and 23F.

In another embodiment the invention also provides the method above of Embodiment 5, wherein the vaccine composition further comprises S. pneumoniae polysaccharide-protein conjugates from serotypes 1, 3, 4, 5, 6A, 6B, 7F, 9V, 14, 18C, 19A, 19F and 23F.

In another embodiment, the invention also provides the method above of Embodiment 5, wherein the vaccine composition further comprises S. pneumoniae polysaccharide-protein conjugates from serotypes 1, 3, 4, 5, 6A, 6B, 7F, 9V, 14, 18C, 19A, 19F, 22F, 23F and 33F.

In another embodiment, the invention also provides the method above of Embodiment 5, wherein the vaccine composition further comprises S. pneumoniae polysaccharide-protein conjugates from serotypes 1, 3, 4, 5, 6A, 6B, 7F, 8, 9V, 10A, 11A, 12F, 14, 15B/C, 18C, 19A, 19F, 22F, 23F and 33F.

In one embodiment of the methods of the present invention, a composition of the present invention is administered as a single inoculation. In another embodiment, the vaccine composition is administered twice, three times or four times or more, adequately spaced apart. For example, the composition may be administered at 1, 2, 3, 4, 5, or 6 month intervals or any combination thereof. The immunization schedule can follow that designated for pneumococcal vaccines. For example, the routine schedule for infants and toddlers against invasive disease caused by S. pneumoniae is 2, 4, 6 and 12-15 months of age. Thus, in a preferred embodiment, the composition is administered as a 4-dose series at 2, 4, 6, and 12-15 months of age.

The compositions of this invention may also include one or more proteins from S. pneumoniae. Examples of S. pneumoniae proteins suitable for inclusion include those identified in International Patent Application Publication Nos. WO 02/083855 and WO 02/053761.

### Formulations

The compositions of the invention can be administered to a subject by one or more method known to a person skilled in the art, such as parenterally, transmucosally, transdermally, intramuscularly, intravenously, intra-dermally, intra-nasally, subcutaneously, intra-peritoneally, and formulated accordingly.

In one embodiment, compositions of the present invention are administered via epidermal injection, intramuscular injection, intravenous, intra-arterial, subcutaneous injection, or intra-respiratory mucosal injection of a liquid preparation. Liquid formulations for injection include solutions and the like.

The composition of the invention can be formulated as single dose vials, multi-dose vials or as pre-filled syringes.

In another embodiment, compositions of the present invention are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

Pharmaceutically acceptable carriers for liquid formulations are aqueous or nonaqueous solutions, suspensions, emulsions or oils. Examples of nonaqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

The pharmaceutical composition may be isotonic, hypotonic or hypertonic. However it is often preferred that a pharmaceutical composition for infusion or injection is essentially isotonic, when it is administrated. Hence, for storage the pharmaceutical composition may preferably be isotonic or hypertonic. If the pharmaceutical composition is hypertonic for storage, it may be diluted to become an isotonic solution prior to administration.

The isotonic agent may be an ionic isotonic agent such as a salt or a non-ionic isotonic agent such as a carbohydrate. Examples of ionic isotonic agents include but are not limited to sodium chloride (NaCl), calcium chloride (CaCl2), potassium chloride (KCl) and magnesium chloride (MgCl2). Examples of non-ionic isotonic agents include but are not limited to mannitol, sorbitol and glycerol.

It is also preferred that at least one pharmaceutically acceptable additive is a buffer. For some purposes, for example, when the pharmaceutical composition is meant for infusion or injection, it is often desirable that the composition comprises a buffer, which is capable of buffering a solution to a pH in the range of 4 to 10, such as 5 to 9, for example 6 to 8.

The buffer may for example be selected from the group consisting of TRIS, acetate, glutamate, lactate, maleate, tartrate, phosphate, citrate, carbonate, glycinate, histidine, glycine, succinate and triethanolamine buffer.

The buffer may furthermore for example be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic; dibasic acids such as aconitic, adipic, ascorbic, carbonic, glutamic, malic, succinic and tartaric, polybasic acids such as citric and phosphoric; and bases such as ammonia, diethanolamine, glycine, triethanolamine, and TRIS.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

The formulations of the invention may also contain a surfactant. Preferred surfactants include, but are not limited to: the polyoxyethylene sorbitan esters surfactants (commonly referred to as the Tweens); copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate.

Preferred amounts of surfactants (% by weight) are: polyoxyethylene sorbitan esters (such as PS80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

The formulation also contains a pH-buffered saline solution. The buffer may, for example, be selected from the group consisting of TRIS, acetate, glutamate, lactate, maleate, tartrate, phosphate, citrate, carbonate, glycinate, histidine, glycine, succinate, HEPES (4-(2-hydroxyethyl)-1-piperazineethanesulfonic acid), MOPS (3-(N-morpholino)propanesulfonic acid), MES (2-(N-morpholino)ethanesulfonic acid) and triethanolamine buffer. The buffer is capable of buffering a solution to a pH in the range of 4 to 10, 5.2 to 7.5, or 5.8 to 7.0. In certain aspect of the invention, the buffer selected from the group consisting of phosphate, succinate, histidine, MES, MOPS, HEPES, acetate or citrate. The buffer may furthermore, for example, be selected from USP compatible buffers for parenteral use, in particular, when the pharmaceutical formulation is for parenteral use. The concentrations of buffer will range from 1 mM to 50 mM or 5 mM to 50 mM. In certain aspects, the buffer is histidine at a final concentration of 5 mM to 50 mM, or succinate at a final concentration of 1 mM to 10 mM. In certain aspects, the histidine is at a final concentration of 20 mM±2 mM.

While the saline solution (i.e., a solution containing NaCl) is preferred, other salts suitable for formulation include but are not limited to, CaCl2, KCl and MgCl2 and combinations thereof. Non-ionic isotonic agents including but not limited to sucrose, trehalose, mannitol, sorbitol and glycerol may be used in lieu of a salt. Suitable salt ranges include, but not are limited to 25 mM to 500 mM or 40 mM to 170 mM. In one aspect, the saline is NaCl, optionally present at a concentration from 20 mM to 170 mM.

In a preferred embodiment, the formulations comprise a L-histidine buffer with sodium chloride.

In another embodiment, the pharmaceutical composition is delivered in a controlled release system. For example, the agent can be administered using intravenous infusion, a transdermal patch, liposomes, or other modes of administration. In another embodiment, polymeric materials are used; e.g. in microspheres in or an implant.

The compositions of this invention may also include one or more proteins from S. pneumoniae. Examples of S. pneumoniae proteins suitable for inclusion include those identified in International Patent Application Publication Nos. WO 02/083855 and WO 02/053761.

### Analytical Methods

### Molecular Weight and Concentration Analysis of Conjugates Using HPSEC/UV/MALS/RI Assay

Conjugate samples are injected and separated by high performance size-exclusion chromatography (HPSEC). Detection is accomplished with ultraviolet (UV), multi-angle light scattering (MALS) and refractive index (RI) detectors in series. Protein concentration is calculated from UV280 using an extinction coefficient. Polysaccharide concentration is deconvoluted from the RI signal (contributed by both protein and polysaccharide) using the dn/dc factors which are the change in a solution's refractive index with a change in the solute concentration reported in mL/g. Average molecular weight of the samples are calculated by Astra software (Wyatt Technology Corporation, Santa Barbara, Calif.) using the measured concentration and light scattering information across the entire sample peak. There are multiple forms of average values of molecular weight for polydispersed molecules. For example, number-average molecular weight Mn, weight-average molecular weight Mw, and z-average molecular weight Mz (Molecules, 2015, 20:10313-10341). Unless specified, the term “molecular weight”, as used throughout the specification, is the weight-average molecular weight.

### Determination of Lysine Consumption in Conjugated Protein as a Measure of the Number of Covalent Attachments Between Polysaccharide and Carrier Protein

The Waters AccQ-Tag amino acid analysis (AAA) is used to measure the extent of conjugation in conjugate samples. Samples are hydrolyzed using vapor phase acid hydrolysis in the Eldex workstation, to break the carrier proteins down into their component amino acids. The free amino acids are derivatized using 6-aminoquinolyl-N-hydroxysuccinimidyl carbamate (AQC). The derivatized samples are then analyzed using UPLC with UV detection on a C18 column. The average protein concentration is obtained using representative amino acids other than lysine. Lysine consumption during conjugation (i.e., lysine loss) is determined by the difference between the average measured amount of lysine in the conjugate and the expected amount of lysine in the starting protein.

### Free Polysaccharide Testing

Free polysaccharide (i.e., polysaccharide that is not conjugated with CRM197) in the conjugate sample is measured by first precipitating free protein and conjugates with deoxycholate (DOC) and hydrochloric acid. Precipitates are then filtered out and the filtrates are analyzed for free polysaccharide concentration by HPSEC/UV/MALS/RI. Free polysaccharide is calculated as a percentage of total polysaccharide measured by HPSEC/UV/MALS/RI.

### Free Protein Testing

Free polysaccharide, polysaccharide-CRM197 conjugate, and free CRM197 in the conjugate samples are separated by capillary electrophoresis in micellar electrokinetic chromatography (MEKC) mode. Briefly, samples are mixed with MEKC running buffer containing 25 mM borate, 100 mM SDS, pH 9.3, and are separated in a preconditioned bare-fused silica capillary. Separation is monitored at 200 nm and free CRM197 is quantified with a CRM197 standard curve. Free protein results are reported as a percentage of total protein content determined by the HPSEC/UV/MALS/RI procedure.

### Polysaccharide Degree of Activation Assay

Conjugation occurs through reductive amination between the activated aldehydes and mainly lysine residues on the carrier protein. The level of activation, as mole of aldehyde per mole of polysaccharide repeat unit, is important to control the conjugation reactions.

In this assay, polysaccharide is derivatized with 2.5 mg/mL thiosemicarbazide (TSC) at pH4.0 to introduce a chromophore (derivatization of activated polysaccharide for serotype 1, 5, 9V uses 1.25 mg/mL TSC). The derivatization reaction was allowed to proceed to reach a plateau. The actual time varies depending on reaction speed of each serotype. TSC-Ps is then separated from TSC and other low molecular weight components by high performance size exclusion chromatography. The signal is detected by UV absorbance at 266 nm. The level of activated aldehyde is calculated either against standard curve injections of Mono-TSC or directly using predetermined extinction coefficient. Mono-TSC is a synthesized thiosemicarbazone derivative of monosaccharide. The aldehyde level is then converted as moles of aldehyde per mole of repeat unit (Ald/RU) using the Ps concentration measured by HPSEC/UV/MALS/RI assay.

Having described various embodiments of the invention with reference to the accompanying description, it is to be understood that the invention is not limited to those precise embodiments, and that various changes and modifications may be effected therein by one skilled in the art without departing from the scope or spirit of the invention as defined in the appended claims.

All publications mentioned herein are incorporated by reference for the purpose of describing and disclosing methodologies and materials that might be used in connection with the present invention.

The following examples illustrate, but do not limit the invention.

### Example 1

### Preparation of Serotype 35B Conjugate

Polysaccharide was dissolved, chemically activated and buffer-exchanged by ultrafiltration. Activated polysaccharide and purified CRM197 were individually lyophilized and re-dissolved in DMSO. Re-dissolved polysaccharide and CRM197 solutions were then combined and conjugated as described below. The resulting conjugate was purified by ultrafiltration prior to a final 0.2-micron filtration. Several process parameters within each step, such as pH, temperature, concentration, and time were controlled to yield conjugates with desired attributes.

### Polysaccharide Oxidation

Purified pneumococcal capsular Ps powder was dissolved in water and 0.45-micron filtered. Dissolved polysaccharide was concentrated and diafiltered against water using a 10 kDa NMWCO tangential flow ultrafiltration membrane.

The polysaccharide solution was then adjusted to 22° C. and pH 5 with a sodium acetate buffer to minimize polysaccharide size reduction due to activation. Polysaccharide activation was initiated with the addition of a 100 mM sodium metaperiodate solution. The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharide was formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were re-dissolved individually in equal volumes of DMSO. The polysaccharide solution was spiked with sodium chloride to a final concentration of 20 mM. The polysaccharide and CRM197 solutions were blended to achieve a polysaccharide concentration of 6.0 g Ps/L and a polysaccharide to CRM197 mass ratio of 3.0. The mass ratio was selected to control the polysaccharide to CRM197 ratio in the resulting conjugate. Conjugation proceeded at 34° C.

Reduction with Sodium Borohydride

Sodium borohydride (2 moles per mole of polysaccharide repeating unit) was added following the conjugation reaction and incubated for 1 hour at 34° C. The batch was diluted into 150 mM sodium chloride, with approximately 0.025% (w/v) polysorbate 20, at approximately 4° C. Potassium phosphate buffer was then added to neutralize the pH. The batch was concentrated and diafiltered at approximately 4° C. against 150 mM sodium chloride, 25 mM potassium phosphate pH 7, using a 30 kD NMWCO tangential flow ultrafiltration membrane.

### Final Filtration and Product Storage

The batch was then concentrated and diafiltered against 10 mM histidine in 150 mM sodium chloride, pH 7.0, with 0.015% (w/v) polysorbate 20, at 4° C. using a 300 kDa NMWCO tangential flow ultrafiltration membrane.

The retentate batch was 0.2 micron filtered (with 0.5 micron prefilter) then diluted with additional 10 mM histidine in 150 mM sodium chloride, pH 7.0 with 0.015% (w/v) polysorbate 20, dispensed into aliquots and frozen at ≤−60° C.

### Example 2

### Formulation of Pneumococcal Conjugate Vaccines

Individual pneumococcal polysaccharide-protein conjugates prepared utilizing different processes as described in the Example(s) above were used for the formulation of monovalent and polyvalent pneumococcal conjugate vaccines.

The PCV21 vaccine drug product used to immunize mice and rabbits was prepared by individually conjugating the CRM197 protein to pneumococcal polysaccharide (PnPs) types (3, 6C, 7F, 8, 9N, 10A, 11A, 12F, 15A, 15C (in the form of deOAc15B), 16F, 17F, 19A, 20, 22F, 23A, 23B, 24F, 31, 33F, 35B) using reductive amination in an aprotic environment (DMSO) and formulated in 20 mM L-Histidine pH 5.8 and 150 mM NaCl and 0.2% w/v Polysorbate-20 (PS-20) at 4.0 μg/mL each serotype for a total polysaccharide concentration of 84.0 μg/mL. The required volume of bulk conjugate needed to obtain the target concentration of total pneumococcal polysaccharide antigen was calculated based on batch volume and concentration of individual bulk polysaccharide concentrations. The individual conjugates were added to a solution of histidine, sodium chloride and PS-20 to produce a 4-fold conjugate blend. The formulation vessel containing the 4-fold conjugate blend is mixed using a magnetic stir bar and then sterile filtered into another vessel. The sterile filtered, 4-fold conjugate blend is then diluted with saline to achieve the desired target polysaccharide and excipient concentrations. The formulations are then filled into glass vials or syringes and stored at 2-8° C.

The monovalent drug product was prepared using pneumococcal polysaccharide 35B-CRM197 conjugate and was formulated in 20 mM histidine pH 5.8 and 150 mM sodium chloride and 0.1% w/v or 0.2% w/v Polysorbate-20 (PS-20) at targeted 4.0 μg/mL pneumococcal polysaccharide antigen. The formulation was prepared with 250 μg [Al]/mL in the form of aluminum phosphate as the adjuvant. The required volume of bulk conjugate needed to obtain the target concentration of individual pneumococcal polysaccharide antigen was calculated based on batch volume and concentration of individual bulk polysaccharide concentration. The individual conjugate was added to a solution of histidine, sodium chloride and PS-20 to produce either a 2-fold or 4-fold conjugate blend. The formulation vessel containing the 2-fold or 4-fold conjugate blend was mixed using a magnetic stir bar and then sterile filtered into another vessel. The sterile filtered, 2-fold or 4-fold conjugate blend was then added to another vessel containing aluminum phosphate adjuvant (APA) to achieve the desired target polysaccharide, excipient and APA concentrations. The formulations are then filled into glass vials or syringes and stored at 2-8° C.

### Example 3

Anti-35B Sera Generated in New Zealand White Rabbits (NZWR) and Mice Immunized with 35B-CRM197 Vaccine Cross Reacts with S. pneumoniae Serotype 29 Bacteria

Adult New Zealand white rabbits (n=3/group) were intramuscularly (IM) immunized with 0.25 ml of 35B-CRM197 vaccine on day 0 and day 14 (alternating sides). 35B-CRM197 vaccine was dosed at 1 μg of 35B polysaccharide conjugated to CRM197 and formulated with 62.5 μg APA per immunization. Sera were collected prior to study start (pre-immune) and on days 14 (post-dose 1, PD1) and 28 (post-dose 2, PD2). NZWRs were observed at least daily by trained animal care staff for any signs of illness or distress. The vaccine formulations in NZWRs were deemed to be safe and well tolerated, as no vaccine-related adverse events were noted. All animal experiments were performed in strict accordance with the recommendations in the Guide for Care and Use of Laboratory Animals of the National Institutes of Health. The NZWR experimental protocol was approved by the Institutional Animal Care and Use Committees at both Merck & Co., Inc and Covance (Denver, Pa.).

Young female CD1 and Swiss Webster (SW) mice (6-8 weeks old, n=10/group) were immunized intramuscularly with 0.1 ml of a 35B-CRM197 vaccine on day 0, day 14, and day 28. 35B-CRM197 vaccine was dosed at 0.4 μg of 35B polysaccharide conjugated to CRM197 with 25 μg APA per immunization. Sera were collected prior to study start (pre-immune) and on day 35 (post-dose 3, PD3). Mice were observed at least daily by trained animal care staff for any signs of illness or distress. The vaccine formulations in mice were deemed to be safe and well tolerated, as no vaccine-related adverse events were noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc.

Rabbit and mice sera were evaluated for anti-35B and anti-29 functional antibody through opsonophagocytosis assays (OPA) based on previously described protocols at www.vaccine.uab.edu and Opsotiter® 3 software owned by and licensed from University of Alabama (UAB) Research Foundation (Burton, R L, Nahm M H, Clin Vaccine Immunol 2006, 13:1004-9; Burton, R L, Nahm M H, Clin Vaccine Immunol 2012, 19:835-41). Rabbit PD2 sera were assayed individually and pre-immune sera were assayed as a pool. Pre-immune and PD3 mouse sera were assayed as a pool.

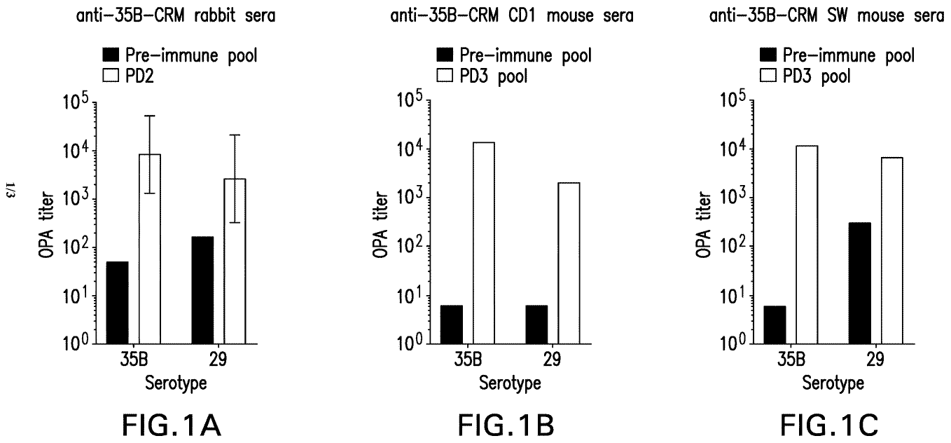

35B-CRM197 vaccine induced high anti-35B OPA titers in rabbits and two strains of mice compared to pre-immune sera. The anti-35B sera also had opsonophagocytic killing activity against serotype 29 strains (FIG. 1A-1C).

### Example 4

Anti-35B Sera Generated in New Zealand White Rabbits Immunized with PCV21 Vaccine Cross Reacts with S. pneumoniae Serotype 29 Bacteria

Adult New Zealand white rabbits (NZWR, n=5/group) were intramuscularly (IM) immunized with 0.1 or 0.25 ml of a 21-valent pneumococcal conjugate vaccine (PCV21/unadjuvanted) on day 0 and day 14 (alternating sides). PCV21 was dosed at 0.4 (group 1) or 1 μg (group 2) of each pneumococcal polysaccharide (3, 6C, 7F, 8, 9N, 10A, 11A, 12F, 15A, 15C, 16F, 17F, 19A, 20A, 22F, 23A, 23B, 24F, 31, 33F, 35B) and all conjugated to CRM197 and unadjuvanted. Sera were collected prior to study start (pre-immune) and on days 14 (PD1) and 28 (PD2). NZWRs were observed at least daily by trained animal care staff for any signs of illness or distress. The vaccine formulations in NZWRs were deemed to be safe and well tolerated, as no vaccine-related adverse events were noted. All animal experiments were performed in strict accordance with the recommendations in the Guide for Care and Use of Laboratory Animals of the National Institutes of Health. The NZWR experimental protocol was approved by the Institutional Animal Care and Use Committees at both Merck & Co., Inc and Covance (Denver, Pa.).

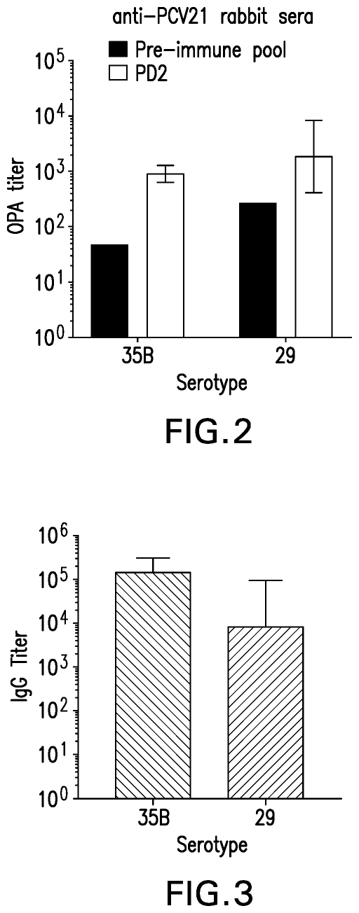

Rabbit sera were evaluated for anti-35B and anti-29 functional antibody through opsonophagocytosis assays (OPA). PCV21 vaccine generated high anti-35B OPA titers at PD2 in rabbits. The anti-PCV21 sera also had opsonophacytic killing activities against serotype 29 strains (FIG. 2).

### Example 5

### Serotype 29 Polysaccharide Partially Inhibits the Anti-35B Sera Opsonophacytic Killing Activity Against Serotype 35B Strain

The hyperimmune sera generated by 35B-CRM197 or PCV21 vaccine were incubated with 100 μg of PnPs15A, or PnPS29, or PnPs35B, or buffer for 30 minutes at room temperature before running the OPA assay. After the PnPs pre-absorption, the sera were evaluated for anti-35B functional antibody through OPA assay.

Pre-absorption with PnPs35B completely inhibited the anti-35B OPA activity in all sera. Pre-absorption with PnPs15A showed no inhibition in the anti-35B OPA activity for anti-35B-CRM197 rabbit sera and 35B-CRM197 CD1 mice sera and had low inhibition for anti-35B-CRM197 SW sera and anti-PCV21 rabbit sera (25%-34%). Pre-absorption with PnPs29 significantly reduced the anti-35B OPA activity in all sera (71%-89%) (TABLE 1). This data demonstrate that PnPs29 can bind to some of the functional antibodies against serotype 35B strain. These antibodies may be induced by the common epitopes shared by PnPs35B and PnPs29 (Geno K A, Nahm M H et al, Clin Microbiol Rev 2015, 28(3):871-899). Based on the data, we hypothesize that PnPs35B maybe partially inhibit the anti-29 OPA activities of hyperimmune sera induced by 29-CRM197 vaccine.

TABLE 1
Relative OPA activity to buffer control after pre-
absorption with PnPs15A, PnPs29, and PnPs35B.

| | Anti-35B- | Anti-35B- | | |
|---|---|---|---|---|
| | CRM197 | CRM197 | Anti-35B- | Anti-PCV21 |
| | rabbit | CD1 mice | CRM197 | rabbit |
| | sera (%) | sera (%) | SW sera (%) | sera (%) |
| Buffer/Buffer | 100 | 100 | 100 | 100 |
| PnPs15A/Buffer | 114 | 92 | 66 | 75 |
| PnPs29/Buffer | 29 | 28 | 11 | 25 |
| PnPs35B/Buffer | 1.4 | 0 | 0 | 0 |

### Example 6

Mice Immunized with the Polysaccharide-Protein Conjugate Serotype 35B-CRM197 Vaccine were Protected from S. pneumoniae Serotype 29 Challenge

Young female CD1 mice (6-8 weeks old, n=10/group) were immunized with 0.1 ml of the 35B-CRM197 vaccine on day 0, day 14, and day 28. 35B-CRM197 vaccine was dosed at 0.4 μg of 35B polysaccharide conjugated to CRM197 with 25 μg APA per immunization. Mice were observed at least daily by trained animal care staff for any signs of illness or distress. The vaccine formulations in mice were deemed to be safe and well tolerated, as no vaccine-related adverse events were noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc.

On day 52 the mice were intratracheally (IT) challenged with S. pneumoniae serotype 29. Exponential phase cultures of S. pneumoniae were centrifuged, washed, and suspended in sterile PBS. Mice were anesthetized with isoflurane prior to challenge. 5×10⁴ cfu of S. pneumoniae serotype 29 in 0.1 ml of PBS was placed in the throat of mice hung upright by their incisors. Aspiration of the bacteria was induced by gently pulling the tongue outward and covering the nostrils. Mice were weighed daily and euthanized if weight loss exceeded 20% of starting weight. Blood was collected at 24, 48 and 72 hours to assess for bacteremia. Mice were observed at least twice daily by trained animal care staff for any signs of illness or distress.

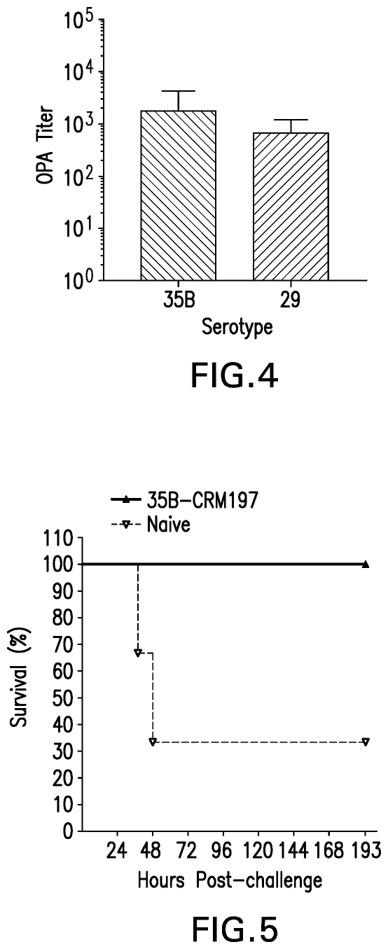

Mouse sera were evaluated for anti-PnPs35B and anti-PnPs29 IgG titers using ELISA as previously described (Chen Z. F. et al, BMC Infectious Disease, 2018, 18: 613). Mouse sera were also evaluated for anti-35B and anti-29 functional antibody through an OPA assay. Mice immunized with 35B-CRM197 vaccine generated both binding antibodies to PnPs35B and PnPs29 (FIG. 3) and functional antibodies to S. pneumoniae serotype 35B strain and serotype 29 bacteria (FIG. 4). Mice immunized with 35B-CRM197 vaccine were also protected from serotype 29 intratracheal challenge (FIG. 5). Mice immunized with 35B-CRM197 vaccine had 100% survival rate compared to 30% survival rate of naïve mice at 8 days post-challenge. These data demonstrate that 35B-CRM197 vaccine can cross-protect the mice from serotype 29 IT challenge. This cross-protection may be mediated through the cross-reactive functional antibodies against serotype 29 strain.

1. (canceled)

2. (canceled)

3. (canceled)

4. (canceled)

5. (canceled)

6. (canceled)

7. (canceled)

8. (canceled)

9. A method for preventing, treating or ameliorating an infection, disease or condition caused by S. pneumoniae serotype 29 in a subject by administering an immunogenic multivalent pneumococcal polysaccharide-protein conjugate vaccine composition which comprises a serotype 35B polysaccharide-protein conjugate to the subject, wherein said vaccine composition does not comprise a serotype 29 polysaccharide-protein conjugate.

10. The method of claim 9, wherein the vaccine composition further comprises S. pneumoniae polysaccharide-protein conjugates from serotypes 4, 6B, 9V, 14, 18C, 19F and 23F.

11. The method of claim 9, wherein the vaccine composition further comprises S. pneumoniae polysaccharide-protein conjugates from serotypes 1, 3, 4, 5, 6A, 6B, 7F, 9V, 14, 18C, 19A, 19F and 23F.

12. The method of claim 9, wherein the vaccine composition further comprises S. pneumoniae polysaccharide-protein conjugates from serotypes 1, 3, 4, 5, 6A, 6B, 7F, 9V, 14, 18C, 19A, 19F, 22F, 23F and 33F.

13. The method of claim 9, wherein the vaccine composition further comprises S. pneumoniae polysaccharide-protein conjugates from serotypes 1, 3, 4, 5, 6A, 6B, 7F, 8, 9V, 10A, 11A, 12F, 14, 15B/C, 18C, 19A, 19F, 22F, 23F and 33F.

14. The method of claim 9, wherein the vaccine composition further comprises S. pneumoniae polysaccharide-protein conjugates from serotypes 8, 10A, 11A, 12F, 15B/C, 22F and 33F.

15. A method for preventing, treating or ameliorating an infection, disease or condition caused by S. pneumoniae serotype 29 in a subject by administering an immunogenic multivalent pneumococcal polysaccharide-protein conjugate vaccine composition which comprises S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B to the subject, wherein said vaccine composition does not comprise a serotype 29 polysaccharide-protein conjugate.

16. A method for preventing, treating or ameliorating an infection, disease or condition caused by S. pneumoniae serotype 29 in a subject by administering an immunogenic multivalent pneumococcal polysaccharide-protein conjugate vaccine composition which consists of S. pneumoniae polysaccharide-protein conjugates from serotypes 3, 6A, 7F, 8, 9N, 10A, 11A, 12F, 15A, 15C, 16F, 17F, 19A, 20A, 22F, 23A, 23B, 24F, 31, 33F and 35B to the subject.
